SEQUENCE LISTING (5 sequences)

SEQ ID NO: 1 - protein, 7 aa, synthetic construct
KGGRAKD

SEQ ID NO: 2 - protein, 7 aa, synthetic construct
DKARGGK

SEQ ID NO: 3 - protein, 20 aa, synthetic construct
ACTGSTQHQGGGSEHGAMEI

SEQ ID NO: 4 - protein, 22 aa, synthetic construct
ACSSSPSKHCGGGSESPLKRQG

SEQ ID NO: 5 - protein, 21 aa, synthetic construct
ACTGSTQHQGGGSESPLKRQG

(19) United States
(12) Patent Application Publication
(10) Pub. No.: US 2024/0216250 A1
(43) Pub. Date: Jul. 4, 2024

(54) COSMETIC PEPTIDES FOR IMPROVING SKIN REJUVENATION

(71) Applicant: Lean Life Sciences, Inc., Tucson, AZ (US)

(72) Inventors: Diana MJ JUNG, Southlake, TX (US); Youngwook Won, Southlake, TX (US); Kyung Won Shin, Baldwin Park, CA (US)

(73) Assignee: Lean Life Sciences, Inc., Tucson, AZ (US)

(21) Appl. No.: 18/432,746

(22) Filed: Feb. 5, 2024

Related U.S. Application Data
(63) Continuation-in-part of application No. PCT/US21/45045, filed on Aug. 6, 2021.

(51) Int. Cl. A61K 8/64; A61K 8/02; A61Q 19/08; C07K 5/083; C07K 5/09

(52) U.S. Cl. CPC A61K 8/64; A61K 8/0208; A61Q 19/08; C07K 5/0806; C07K 5/0817

(57) ABSTRACT

Provided herein are cosmetic compositions and methods for improving skin rejuvenation. In particular, provided herein are peptides and uses of such peptides in the reduction of a level of glucose, fructose, sucrose and/or polysaccharide in adipocytes or skin cells and/or prevention of collagen degradation.

### CROSS REFERENCE TO RELATED APPLICATIONS

This application is a Rule 53(b) Continuation-In-Part of International Application No. PCT/US2021/045045 filed on Aug. 6, 2021.

### INCORPORATION BY REFERENCE OF SEQUENCE LISTING

The content of the electronically submitted sequence listing, file name: A295368_sequence listing as filed; size: 5,188 bytes; and date of creation: Jan. 19, 2024, filed herewith, is incorporated herein by reference in its entirety.

### FIELD

Provided herein are cosmetic compositions and methods for rejuvenating skin through anti-aging, detoxification, anti-glycation, collagen regeneration, and/or improving elasticity and/or youthfulness of skin. In particular, provided herein are peptides and uses of such peptides for cosmetic compositions. Such peptides exert anti-aging, detoxification, and/or collagen regeneration through reduction of glucose, fructose, sucrose, and other polysaccharides levels (anti-glycation) in skin cells.

### BACKGROUND

According to Allied Market Research, the global cosmetics market size was valued at $380.2 billion in 2019, and is projected to reach $463.5 billion by 2027 registering a CAGR of 5.3% from 2021 to 2027. The functional cosmetic product is one of the fastest growing cosmetic products in the cosmetic market thanks to research on the health and cosmetic benefits of various biochemical products.

Collagen is the most abundant protein in skin and a major component of skin, consisting up to 75-80% of skin components. Collagen plays a critical role in maintaining skin tension, elasticity and hydration. The environment and aging reduce the body's ability to produce collagen. Protein glycation is involved in a general process of aging, especially long-lived proteins, such as structural collagen. Thinner and wrinkled skin, the typical signs of normal aging, are the consequence of reduced collagen. Protein glycation contributes to the skin aging through deterioration of the existing collagen by forming inter- and intra-molecular crosslinking. Accelerated skin aging is especially noticeable in diabetic patients, where overall glucose level is elevated. For diabetic patients, anti-glycation agents are available. However, glycation is significantly associated with skin aging in not only patients with diabetes, but also non-diseased individuals. The present inventors discovered safe anti-glycation substances as an ingredient of cosmetic compositions.

Glycation not only influences the properties of collagen and the extracellular matrix but also matrix-to-cell interactions. The extracellular matrix alters the characteristics of resident cells in skin, including migration, growth, proliferation, differentiation, and gene expression. Thus, physical changes in matrix components, such as nonenzymatic glycation of collagen, may affect such behaviors of skin cells. Collagen and elastin, the two major structural proteins of the extracellular matrix, are subject to those molecular changes and can be stimulated to form cascade cross-linking and side-chain modifications.

Polysaccharides, including fructose, are known to be involved in the abovementioned alterations. Elevated levels of fructose, therefore, facilitate intra- and inter-molecular crosslinking in collagen, both of which in turn reduce skin's elasticity and softness, the hallmarks of youthfulness of skin (Levi et al., The Journal of Nutrition, Volume 128, Issue 9, September 1998, Pages 1442-1449). High-sugar-contained diets and increased consumption of fructose in the modern diet can negatively affect the elasticity and youthfulness of the skin. Thus, reduced consumption of fructose or facilitated metabolism of fructose in skin may contribute to the maintenance of elasticity and youthfulness of skin and ultimately decelerates the aging. Therefore, functional cosmetic products reducing the fructose level can help prevent aging of skin.

However, effective cosmetic compositions in this regard have not been proposed thus far.

### SUMMARY

Provided herein is a cosmetic composition comprising one or more (e.g., a combination of two or more, three or more, etc.) peptides having an amino acid sequence selected from, for example, KGGRAKD (SEQ ID NO:1), KGG, GGR, GRA, RAK, AKD, DKA, KAR, ARG, RGG, GGK, DKARGGK (SEQ ID NO:2), ACTGSTQHQGGGSEHGAMEI (SEQ ID NO: 3), ACSSSPSKHCGGGSESPLKRQG (SEQ ID NO: 4), and ACTGSTQHQGGGSESPLKRQG (SEQ ID NO: 5) or a variant or mimetic thereof. In some embodiments, the cosmetic composition comprises KGGRAKD (SEQ ID NO:1) and one or more of KGG, GRA, GGR, RAK, or AKD. In some embodiments, the peptide is cyclized (e.g., via the addition of a cysteine to each end of the peptide). In certain embodiments, the peptide is modified. In some embodiments, the composition comprises a cosmetically acceptable topical carrier. In some embodiments, the composition further comprises one or more additional cosmetic agents. In another embodiment, the peptide is RAK or GGR. In some embodiments, the cosmetic composition is for use in reducing a glucose, fructose, sucrose, and other polysaccharides level in cells including skin cells and adipocytes. In another embodiment, the cosmetic composition is for use in preventing collagen degradation, lipid (fat accumulation), and aging. In some embodiments, the cosmetic composition is for use in improving elasticity and/or youthfulness of skin leading to rejuvenation of skin (cells). In another embodiment, the cosmetic composition is formulated in the form of a cream, a lotion, a sunscreen product, an ointment, a spray, a powder, a tanning product, a colored cosmetic product, an ointment, and/or any types that are applicable to skin.

Additional embodiments provide a patch comprising a cosmetic composition comprising one or more peptides having an amino acid sequence selected from the group consisting of KGGRAKD (SEQ ID NO:1), KGG, GGR, GRA, RAK, AKD, DKA, KAR, ARG, RGG, GGK, DKARGGK (SEQ ID NO:2), ACTGSTQHQGGGSEHGAMEI (SEQ ID NO: 3), ACSSSPSKHCGGGSESPLKRQG (SEQ ID NO: 4), and ACTGSTQHQGGGSESPLKRQG (SEQ ID NO: 5) or a variant or mimetic thereof. In another embodiment, the patch is in the form of a microneedle patch, or a hyaluronic acid patch.

Further embodiments provide a method of improving skin rejuvenation, comprising applying the cosmetic composition comprising one or more peptides having an amino acid sequence selected from the group consisting of KGGRAKD (SEQ ID NO:1), KGG, GGR, GRA, RAK, AKD, DKA, KAR, ARG, RGG, GGK, DKARGGK (SEQ ID NO:2), ACTGSTQHQGGGSEHGAMEI (SEQ ID NO: 3), ACSSSPSKHCGGGSESPLKRQG (SEQ ID NO: 4), and ACTGSTQHQGGGSESPLKRQG (SEQ ID NO: 5) or a variant or mimetic thereof to a skin of a subject in need thereof. The improving skin rejuvenation may include anti-aging, detoxification, anti-glycation, collagen regeneration, and/or improving elasticity and/or youthfulness of skin. In another embodiment, the applying the cosmetic composition reduces a level of glucose, fructose, sucrose and/or polysaccharide in adipocytes or skin cells of the subject. In another embodiment, the applying the cosmetic composition prevents collagen degradation in the subject.

Additional embodiments provide a method of improving skin rejuvenation, comprising applying a patch comprising a cosmetic composition comprising one or more peptides having an amino acid sequence selected from the group consisting of KGGRAKD (SEQ ID NO:1), KGG, GGR, GRA, RAK, AKD, DKA, KAR, ARG, RGG, GGK, DKARGGK (SEQ ID NO:2), ACTGSTQHQGGGSEHGAMEI (SEQ ID NO: 3), ACSSSPSKHCGGGSESPLKRQG (SEQ ID NO: 4), and ACTGSTQHQGGGSESPLKRQG (SEQ ID NO: 5) or a variant or mimetic thereof to a skin of a subject in need thereof. In another embodiment, the applying the patch reduces a level of glucose, fructose, sucrose and/or polysaccharide in adipocytes or skin cells of the subject. In another embodiment, the applying the patch prevents collagen degradation in the subject, detoxifies and rejuvenates.

Additional embodiments are described herein.

### BRIEF DESCRIPTION OF THE DRAWINGS

FIG. 1: Relative fructose levels in adipocytes upon treatment of the peptides.

FIG. 2: ATS/prohibitin binding determined by immunoprecipitation and western blot (A of FIG. 2). Location and degree of prohibitin expression in pre- or mature adipocytes. PM, plasma membrane; Cyt, Cytoplasm (B of FIG. 2). Confocal micrographs showing the location of prohibitin in mature- or pre-adipocytes (C of FIG. 2).

FIG. 3: Representative photomicrographs showing Oil Red O-stained mature adipocytes treated with peptides at 100 μM (Day 21). H: Quantitative analysis of the lipids accumulated in the adipocytes (Day 21). Control absorbance: 0.526 (Red line). I: Relative cell counts on day 21.

FIG. 4: IP Preobese Body Weight. Relative body weight measurements for IP injected pre-obese mice. I-J: Body weight change. Changes in body weight compared using HFD control group as baseline value A1-F1: GTT. Blood glucose levels of peptide groups at endpoint, post-glucose injection (after 16 h fasting). A2-F2: ITT. Blood glucose levels of peptide groups at endpoint, post-insulin injection (after 6 h fasting).

FIG. 5: SC Pre-obese Body Weight. Relative body weight measurements for SC injected pre-obese mice. G-H: Body weight change. Changes in body weight compared using HFD control group as baseline value. A1-F1: GTT. Blood glucose levels of peptide groups at endpoint, post-glucose injection (after 16 h fasting). A2-F2: ITT. Blood glucose levels of peptide groups at endpoint, post-insulin injection (after 6 h fasting).

FIG. 6: IP Post-obese Body Weight. Relative body weight measurements for IP injected post-obese mice. G-H: Body weight change. Changes in body weight compared using HFD control group as baseline value. A1-F1: GTT. Blood glucose levels of peptide groups at endpoint, post-glucose injection (after 16 h fasting). A2-F2: ITT. Blood glucose levels of peptide groups at endpoint, post-insulin injection (after 6 h fasting).

FIG. 7: SC Post-obese Body Weight. Relative body weight measurements for SC injected post-obese mice. G-H: Body weight change. Changes in body weight compared using HFD control group as baseline value. A1-D1,F1: GTT. Blood glucose levels of peptide groups at endpoint, post-glucose injection (after 16 h fasting). A2-D2,F2: ITT. Blood glucose levels of peptide groups at endpoint, post-insulin injection (after 6 h fasting).

FIG. 8: Oral/Feeding Pre-obese Body Weight. Relative body weight measurements for orally administered pre-obese mice. A1-F1: GTT. Blood glucose levels at endpoint, post-glucose injection (after 16 h fasting). A2-F2: ITT. Blood glucose levels at endpoint, post-insulin injection (after 6 h fasting).

FIG. 9: Oral/Feeding Post-obese Body Weight. Relative body weight measurements for orally administered post-obese mice. A1-F1: GTT. Blood glucose levels at endpoint, post-glucose injection (after 16 h fasting). A2-F2: ITT. Blood glucose levels at endpoint, post-insulin injection (after 6 h fasting).

FIG. 10. Histological analysis fat pads. An automated system detected and counted adipocytes (yellow line). Adipocyte number and size: average of 3 hpf images from 2 mice (total 6 images/group).

FIG. 11: Collagen and Fibronectin levels in skin cells upon treatment of ACTGSTQHQGGGSESPLKRQG (SEQ ID NO: 5) at varying concentrations.

FIG. 12: DIO mice via intraperitoneal (IP) and subcutaneous (SC) injections.

FIG. 13: DIO model generation scheme and injection schedule.

### Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide” is a reference to one or more peptides and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine (“tBuG”), 2,4-di aminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers to a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (synthetic) sequence.

As used herein, the term “mutant peptide” or “variant peptide” refers to a peptide having a distinct amino acid sequence from the most common variant occurring in nature, referred to as the “wild-type” sequence. A mutant peptide may be a subsequence of a mutant protein or polypeptide (e.g., a subsequence of a naturally-occurring protein that is not the most common sequence in nature) or may be a peptide that is not a subsequence of a naturally occurring protein or polypeptide.

As used herein, the term “artificial peptide” or “artificial polypeptide” refers to a peptide or polypeptide having a distinct amino acid sequence from those found in natural peptides and/or proteins. An artificial protein is not a subsequence of a naturally occurring protein, either the wild-type (i.e., most abundant) or mutant versions thereof. For example, an artificial peptide or polypeptide is not a subsequence of naturally occurring protein (e.g., ATS protein). An artificial peptide or polypeptide may be produced or synthesized by any suitable method (e.g., recombinant expression, chemical synthesis, enzymatic synthesis, etc.).

The terms “peptide mimetic” or “peptidomimetic” refer to a peptide-like molecule that emulates a sequence derived from a protein or peptide. A peptide mimetic or peptidomimetic may contain amino acids and/or non-amino acid components. Examples of peptidomimitecs include chemically modified peptides, peptoids (side chains are appended to the nitrogen atom of the peptide backbone, rather than to the α-carbons), β-peptides (amino group bonded to the β carbon rather than the α carbon), etc.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (histidine (H), lysine (K), and arginine (R)); polar negative (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs. Non-conservative substitutions may involve the exchange of a member of one class for a member from another class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a human subject that is being treated for a disease or condition.

As used herein, the term “effective amount” refers to the amount of a sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “treatment” means an approach to obtaining a beneficial or intended clinical result. The beneficial or intended clinical result may include alleviation of symptoms, a reduction in the severity of the disease, inhibiting a underlying cause of a disease or condition, steadying diseases in a non-advanced state, delaying the progress of a disease, and/or improvement or alleviation of disease conditions.

As used herein, the term “cosmetic composition” refers to the combination of an active agent (e.g., ATS-derived peptide) with a carrier, inert or active, making the composition especially suitable for cosmetic use in vitro, in vivo or ex vivo. The terms “cosmetically acceptable” as used herein, refers to compositions that do not substantially produce adverse reactions, e.g., toxic, allergic, or immunological reactions, when administered to a subject.

As used herein, the term “cosmetically acceptable carrier” or “cosmetically acceptable topical carrier” refer to any of the standard cosmetical carriers including, but not limited to, a water-in-oil emulsion, cream, liquid, gel, oil, paste, ointment, suspension, foam, lotion, oil-in-water emulsion, water-in-oil-in-water emulsion, water-in-silicone emulsion, spray or serum carrier. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

As used herein, the term “patch” refers to a form of transdermal delivery that is applied on the surface of the skin. The patch may include an adhesive skin patch, a face mask, a microneedle patch, and a hyaluronic acid patch, etc. The patch may include a front side which is to be applied on the surface of the skin, and where a cosmetic composition is provided, and a rear side. The microneedle patch refers to a patch where microneedles are provided on one side thereof to penetrate the skin's surface for transdermal administration of the active ingredient (e.g., cosmetic composition). The microneedles may be classified as solid microneedles for the pretreatment of skin, coated microneedles with water-soluble formulations, dissolving microneedles without residual fragments, and hollow microneedles for liquid formulations. See Yang et al., Acta Pharmaceutica Sinica B, Volume 9, Issue 3, 2019, Pages 469-483, incorporated herein by reference in its entirety. In addition, there are several other types of microneedles such as a disposable-manner microneedle made of carboxy-methyl-cellulose, a multi-round responsive microneedle made of alginate, a temperature responsive microneedle made of vinyl pyrrolidone, a glucose responsive microneedle made of hyaluronic acid, a pH responsive microneedle made of hyaluronic acid, a swelling-shrinking microneedle made of hydrogel, a water-soluble microneedle made of dextrin, etc. Id. The solid microneedles can be designed as skin pretreatment for producing large pores to deliver the composition. After the pores were formed, topical formulations (ointment, gel, and lotion) applying to treat skin are able to be transported into the dermis through the pores. Subsequently, they can be distributed in all parts of the body by systemic circulation. The coated microneedles may have two main functions. One is to pierce skin and the other is to deliver a desired composition applying on the surface of microneedle. The dissolving microneedles manufactured from safe materials, such as biodegradable polymers and natural polymers, can control the release of the active ingredient embedded in the polymer. That is, dissolving microneedles controlling the release of encapsulated cosmetic agents are painless and safe in the application of cosmetic use.

As used herein, the term “adipocyte” refers to a cell existing in or derived from fat tissue which is terminally differentiated. In their differentiated state, adipocytes assume a rounded morphology associated with cytoskeletal changes and loss of mobility. They further accumulate lipid as multiple small vesicles that later coalesce into a single, large lipid droplet displacing the nucleus. The term “human adipocyte” refers to an adipocyte existing in or isolated from human fat tissue. Adipocytes play a critical role in energy homeostasis. They synthesize and store lipids when nutrients are plentiful, and release fatty acids into the circulation when nutrients are required. Numerous adipogenic genes are expressed in functional adipocytes, whereas they are not expressed in preadipocytes in which lipid are not accumulated either. Adipocyte development has been extensively studied in cell culture as well as in animal models. There are several lines of evidence supporting that adipose tissue dysfunction plays an important role in the pathogenesis of type II diabetes mellitus, i.e. failure of adipocyte differentiation is a predisposition to developing diabetes, (see, e.g., Danforth (2000) Nature Genetics 26: 13).

As used herein, the term “skin cell” refers to a cell derived from the skin of a subject.

### DETAILED DESCRIPTION

The present disclosure provides a cosmetic composition comprising one or more peptides having an amino acid sequence selected from the group consisting of KGGRAKD (SEQ ID NO:1), KGG, GGR, GRA, RAK, AKD, DKA, KAR, ARG, RGG, GGK, DKARGGK (SEQ ID NO:2), ACTGSTQHQGGGSEHGAMEI (SEQ ID NO: 3), ACSSSPSKHCGGGSESPLKRQG (SEQ ID NO: 4), and ACTGSTQHQGGGSESPLKRQG (SEQ ID NO: 5) or a variant or mimetic thereof. In some embodiments, provided herein are cosmetic compositions comprising nucleic acids encoding the above peptides, proteins and polypeptides herein, molecular complexes of the foregoing, etc. for reducing a level of glucose, fructose, sucrose and/or polysaccharide in adipocytes or skin cells, preventing collagen degradation, and/or improving skin rejuvenation. The improving skin rejuvenation may include anti-aging, detoxification, anti-glycation, collagen regeneration, and/or improving elasticity and/or youthfulness of skin. In some embodiments, compositions comprise multiple different peptides selected from the group consisting of KGGRAKD (SEQ ID NO:1), KGG, GGR, GRA, RAK, AKD, DKA, KAR, ARG, RGG, GGK, DKARGGK (SEQ ID NO:2), ACTGSTQHQGGGSEHGAMEI (SEQ ID NO: 3), ACSSSPSKHCGGGSESPLKRQG (SEQ ID NO: 4), and ACTGSTQHQGGGSESPLKRQG (SEQ ID NO: 5).

In some embodiments, the cosmetic composition is used to reduce a level of glucose, fructose, sucrose and/or polysaccharide in adipocytes or skin cells. In another embodiment, the cosmetic composition is used to prevent collagen degradation. The cosmetic composition may also be used to improve skin rejuvenation. The improving skin rejuvenation may include anti-aging, detoxification, anti-glycation, collagen regeneration, and/or improving elasticity and/or youthfulness of skin. In particular, the peptide of the present disclosure prevents fat cell accumulation through its reductive effect on fructose levels (or glucose, sucrose or other polysaccharides level) in the body, which in turn impedes fatty acid synthesis. This is particularly significant during the ongoing obesity epidemic and in a modern world of high-sugar diets and increased consumption of fructose. Maintaining low fructose levels (or glucose, sucrose or other polysaccharide level) has a dual beneficial effect and not only reduces fat cell accumulation but also helps prevent collagen degradation. Collagen plays an important role in retaining healthy and smooth skin after long-time, inevitable exposure to damaging factors, such as sunlight and pollution. Therefore, sustaining high levels of collagen and low levels of fructose contribute to the elasticity and youthfulness of the skin and ultimately decelerates the aging process.

In some embodiments, the peptide of the present disclosure is cyclized. In particular, the peptide of the present disclosure may be cyclized via addition of a cysteine to each end of the peptide. In another embodiment, the peptide may be modified (e.g., substitution, deletion, or addition of standard amino acids; chemical modification; etc.) as long as it provides an effect of reducing a fructose level (or glucose, sucrose or other polysaccharides level) in adipocytes or skin cells, preventing collagen degradation, and/or improving elasticity and/or youthfulness of skin.

In some embodiments, a peptide provided herein is an artificial, not naturally-occurring, sequence. In some embodiments, a peptide described herein is prepared by methods known to those of ordinary skill in the art. For example, the peptide can be synthesized using solid phase polypeptide synthesis techniques (e.g. Fmoc or Boc chemistry). Alternatively, the peptide can be produced using recombinant DNA technology (e.g., using bacterial or eukaryotic expression systems). Further, a peptide may be expressed within a subject (e.g., following administration of an appropriate vector). Accordingly, to facilitate such methods, provided herein are genetic vectors (e.g., plasmids, viral vectors (e.g. AAV), etc.) comprising a sequence encoding the peptide, as well as host cells comprising such vectors. Furthermore, provided herein are the peptide produced via such methods.

In some embodiments, the administration of compositions described herein (e.g., ATS (adipocyte-targeting sequence (SEQ ID NO: 1)) and ATS-derived peptides), variants and mimetics thereof, nucleic acids encoding such peptides, etc.) is provided. In some embodiments, provided herein is the administration of bioactive agents which reduce the fructose level (or glucose, sucrose or other polysaccharides level), prevent collagen degradation and/or improve skin rejuvenation. The improving skin rejuvenation may include anti-aging, detoxification, anti-glycation, collagen regeneration, and/or improving elasticity and/or youthfulness of skin.

Embodiments are not limited to the specific sequences listed herein. In some embodiments, peptides meeting limitations described herein and having substitutions not explicitly described are within the scope of embodiments here. In some embodiments, the peptides described herein are further modified (e.g., substitution, deletion, or addition of standard amino acids; chemical modification; etc.). Modifications that are understood in the field include N-terminal modification, C-terminal modification (which protects the peptide from proteolytic degradation), alkylation of amide groups, hydrocarbon “stapling” (e.g., to stabilize conformations). In some embodiments, the peptides/polypeptides described herein may be modified by conservative residue substitutions, for example, of the charged residues (K to R, R to K, D to E and E to D). Modifications of the terminal carboxy group include, without limitation, the amide, lower alkyl amide, constrained alkyls (e.g. branched, cyclic, fused, adamantyl) alkyl, dialkyl amide, and lower alkyl ester modifications. Lower alkyl is C1-C4 alkyl. Furthermore, one or more side groups, or terminal groups, may be protected by protective groups known to the ordinarily-skilled peptide chemist. The α-carbon of an amino acid may be mono- or dimethylated.

In some embodiments, peptides are provided comprising: (i) one or more of the amino acid residues in the peptide are D-enantiomers, (ii) an N-terminally acetyl group, (iii) a deamidated C-terminal group, (iv) one or more unnatural amino acids, (v) one or more amino acid analogs, and/or (vi) one or more peptoid amino acids. In some embodiments, the peptide or an amino acid therein comprises a modification selected from the group consisting of phosphorylation, glycosylation, ubiquitination, S-nitrosylation, methylation, N-acetylation, lipidation, lipoylation, deimination, eliminylation, disulfide bridging, isoaspartate formation, racemization, glycation; carbamylation, carbonylation, isopeptide bond formation, sulfation, succinylation, S-sulfonylation, S-sulfinylation, S-sulfenylation, S-glutathionylation, pyroglutamate formation, propionylation, adenylylation, nucleotide addition, iodination, hydroxylation, malonylation, butyrylation, amidation, C-terminal amidation, de-amidation, alkylation, acylation, biotinylation, carbamylation, oxidation, and pegylation.

In some embodiments, any embodiments described herein may comprise mimetics corresponding to ATS-derived peptide and/or variants thereof, with various modifications that are understood in the field. In some embodiments, residues in the peptide sequences described herein may be substituted with amino acids having similar characteristics (e.g., hydrophobic to hydrophobic, neutral to neutral, etc.) or having other desired characteristics (e.g., more acidic, more hydrophobic, less bulky, more bulky, etc.). In some embodiments, non-natural amino acids (or naturally-occurring amino acids other than the standard 20 amino acids) are substituted in order to achieve desired properties.

In some embodiments, residues having a side chain that is positively charged under physiological conditions, or residues where a positively-charged side chain is desired, are substituted with a residue including, but not limited to: lysine, homolysine, 8-hydroxylysine, homoarginine, 2,4-diaminobutyric acid, 3-homoarginine, D-arginine, arginal (—COOH in arginine is replaced by —CHO), 2-amino-3-guanidinopropionic acid, nitroarginine (N(G)-nitroarginine), nitrosoarginine (N(G)-nitrosoarginine), methylarginine (N-methyl-arginine), ε-N-methyllysine, allo-hydroxylysine, 2,3-diaminopropionic acid, 2,2′-diaminopimelic acid, ornithine, sym-dimethylarginine, asym-dimethylarginine, 2,6-diaminohexinic acid, p-aminobenzoic acid and 3-aminotyrosine and, histidine, 1-methylhistidine, and 3-methylhistidine.

A neutral residue is a residue having a side chain that is uncharged under physiological conditions. A polar residue preferably has at least one polar group in the side chain. In some embodiments, polar groups are selected from hydroxyl, sulfhydryl, amine, amide and ester groups or other groups which permit the formation of hydrogen bridges.

In some embodiments, residues having a side chain that is neutral/polar under physiological conditions, or residues where a neutral side chain is desired, are substituted with a residue including, but not limited to: asparagine, cysteine, glutamine, serine, threonine, tyrosine, citrulline, N-methylserine, homoserine, allo-threonine and 3,5-dinitro-tyrosine, and β-homoserine.

Residues having a non-polar, hydrophobic side chain are residues that are uncharged under physiological conditions, preferably with a hydropathy index above 0, particularly above 3. In some embodiments, non-polar, hydrophobic side chains are selected from alkyl, alkylene, alkoxy, alkenoxy, alkylsulfanyl and alkenylsulfanyl residues having from 1 to 10, preferably from 2 to 6, carbon atoms, or aryl residues having from 5 to 12 carbon atoms. In some embodiments, residues having a non-polar, hydrophobic side chain are, or residues where a non-polar, hydrophobic side chain is desired, are substituted with a residue including, but not limited to: leucine, isoleucine, valine, methionine, alanine, phenylalanine, N-methylleucine, tert-butylglycine, octylglycine, cyclohexylalanine, ß-alanine, 1-aminocyclohexylcarboxylic acid, N-methylisoleucine, norleucine, norvaline, and N-methylvaline.

In some embodiments, peptide and polypeptides are isolated and/or purified (or substantially isolated and/or substantially purified). Accordingly, in such embodiments, peptides and/or polypeptides are provided in substantially isolated form. In some embodiments, peptides and/or polypeptides are isolated from other peptides and/or polypeptides as a result of solid phase peptide synthesis, for example. Alternatively, peptides and/or polypeptides can be substantially isolated from other proteins after cell lysis from recombinant production. Standard methods of protein purification (e.g., HPLC) can be employed to substantially purify peptides and/or polypeptides. In some embodiments, the present invention provides a preparation of peptides and/or polypeptides in a number of formulations, depending on the desired use. For example, where the polypeptide is substantially isolated (or even nearly completely isolated from other proteins), it can be formulated in a suitable medium solution for storage (e.g., under refrigerated conditions or under frozen conditions). Such preparations may contain protective agents, such as buffers, preservatives, cryprotectants (e.g., sugars such as trehalose), etc. The form of such preparations can be solutions, gels, etc. In some embodiments, peptides and/or polypeptides are prepared in lyophilized form. Moreover, such preparations can include other desired agents, such as small molecules or other peptides, polypeptides or proteins. Indeed, such a preparation comprising a mixture of different embodiments of the peptides and/or polypeptides described here may be provided.

In some embodiments, provided herein are peptidomimetic versions of the peptide sequences described herein or variants thereof. In some embodiments, a peptidomimetic is characterized by an entity that retains the polarity (or non-polarity, hydrophobicity, etc.), three-dimensional size, and functionality (bioactivity) of its peptide equivalent but wherein all or a portion of the peptide bonds have been replaced (e.g., by more stable linkages). In some embodiments, ‘stable’ refers to being more resistant to chemical degradation or enzymatic degradation by hydrolytic enzymes. In some embodiments, the bond which replaces the amide bond (e.g., amide bond surrogate) conserves some properties of the amide bond (e.g., conformation, steric bulk, electrostatic character, capacity for hydrogen bonding, etc.). Cyclization (head-to-tail, head/tail-to-side-chain, and/or side-chain-to-side-chain) enhances peptide stability and permeability by introducing conformation constraint, thereby reducing peptide flexibility, and a cyclic enkephalin analog is highly resistant to enzymatic degradation. Chapter 14 of “Drug Design and Development”, Krogsgaard, Larsen, Liljefors and Madsen (Eds) 1996, Horwood Acad. Publishers provides a general discussion of techniques for the design and synthesis of peptidomimetics and is herein incorporated by reference in its entirety. Suitable amide bond surrogates include, but are not limited to: N-alkylation (Schmidt, R. et al., Int. J. Peptide Protein Res., 1995, 46,47; herein incorporated by reference in its entirety), retro-inverse amide (Chorev, M. and Goodman, M., Acc. Chem. Res, 1993, 26, 266; herein incorporated by reference in its entirety), thioamide (Sherman D. B. and Spatola, A. F. J. Am. Chem. Soc., 1990, 112, 433; herein incorporated by reference in its entirety), thioester, phosphonate, ketomethylene (Hoffman, R. V. and Kim, H. O. J. Org. Chem., 1995, 60, 5107; herein incorporated by reference in its entirety), hydroxymethylene, fluorovinyl (Allmendinger, T. et al., Tetrahydron Lett., 1990, 31, 7297; herein incorporated by reference in its entirety), vinyl, methylencamino (Sasaki, Y and Abe, J. Chem. Pharm. Bull. 1997 45, 13; herein incorporated by reference in its entirety), methylenethio (Spatola, A. F., Methods Neurosci, 1993, 13, 19; herein incorporated by reference in its entirety), alkane (Lavielle, S. et. al., Int. J. Peptide Protein Res., 1993, 42, 270; herein incorporated by reference in its entirety) and sulfonamido (Luisi, G. et al. Tetrahedron Lett. 1993, 34, 2391; herein incorporated by reference in its entirety).

As well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Suitable peptidomimetics include reduced peptides where the amide bond has been reduced to a methylene amine by treatment with a reducing agent (e.g. borane or a hydride reagent such as lithium aluminum-hydride); such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA (1994) 91, 11138-11142; herein incorporated by reference in its entirety.

In various embodiments, the peptides disclosed herein are derivatized by conjugation to one or more polymers or small molecule substituents.

In certain of these embodiments, the peptides described herein (or variants thereof) are derivatized by coupling to polyethylene glycol (PEG). Coupling may be performed using known processes. Sec, Int. J. Hematology, 68:1 (1998); Bioconjugate Chem., 6:150 (1995); and Crit. Rev. Therap. Drug Carrier Sys., 9:249 (1992) all of which are incorporated herein by reference in their entirety. Those skilled in the art, therefore, will be able to utilize such well-known techniques for linking one or more polyethylene glycol polymers to the peptides and polypeptides described herein. Suitable polyethylene glycol polymers typically are commercially available or may be made by techniques well known to those skilled in the art. The polyethylene glycol polymers preferably have molecular weights between 500 and 20,000 and may be branched or straight chain polymers.

The attachment of a PEG to a peptide or polypeptide described herein can be accomplished by coupling to amino, carboxyl or thiol groups. These groups will typically be the N- and C-termini and on the side chains of such naturally occurring amino acids as lysine, aspartic acid, glutamic acid and cysteine. Since the peptides and polypeptides of the present disclosure can be prepared by solid phase peptide chemistry techniques, a variety of moieties containing diamino and dicarboxylic groups with orthogonal protecting groups can be introduced for conjugation to PEG.

The present disclosure also provides for conjugation of the peptides described herein (variants thereof) to one or more polymers other than polyethylene glycol.

In some embodiments, the peptides described herein are derivatized by conjugation or linkage to, or attachment of, polyamino acids (e.g., poly-his, poly-arg, poly-lys, etc.) and/or fatty acid chains of various lengths to the N- or C-terminus or amino acid residue side chains. In certain embodiments, the peptides and polypeptides described herein are derivatized by the addition of polyamide chains, particularly polyamide chains of precise lengths, as described in U.S. Pat. No. 6,552,167, which is incorporated by reference in its entirety. In yet other embodiments, the peptides and polypeptides are modified by the addition of alkylPEG moieties as described in U.S. Pat. Nos. 5,359,030 and 5,681,811, which are incorporated by reference in their entireties.

In select embodiments, the peptides described herein (or variants thereof) are derivatized by conjugation to polymers that include albumin and gelatin. See, Gombotz and Pettit, Bioconjugate Chem., 6:332-351, 1995, which is incorporated herein by reference in its entirety.

In further embodiments, the peptides described herein are conjugated or fused to immunoglobulins or immunoglobulin fragments, such as antibody Fc regions.

In some embodiments, the cosmetic compositions described herein (e.g., comprising the peptides described herein find use in the improvement of skin conditions. In some embodiments, the compositions are applied to the skin of a subject. In certain embodiments, the subject is an adult. In other embodiments, the subject is a child. In another embodiments, the subject is overweight or obese.

In various embodiments, the peptides described herein are applied to the skin of the subject in an amount, on a schedule, and for a duration sufficient to decrease the fructose level (or glucose, sucrose or other polysaccharides level) in adipocytes or skin cells of the subject, prevent collagen degradation in the subject, or improve elasticity and/or youthfulness of the skin of the subject.

In some embodiments, provided herein are cosmetic compositions comprising of one or more ATS or ATS derived peptides or variants thereof and a cosmetically acceptable carrier. Any carrier which can supply an active peptide or polypeptide (e.g., without destroying the peptide or polypeptide within the carrier) is a suitable carrier, and such carriers are well known in the art. In some embodiments, compositions may be formulated in the form of a cream, a lotion, a sunscreen product, a spray, a powder, a tanning product, and a colored cosmetic product. The cosmetic composition may also be in the form of foundations, concealers, blushes, rouges, lipsticks, lip stains, lip glosses, mascaras, eyeshadows and eyeliners.

The cosmetic compositions of the present disclosure may further contain at least one other cosmetically acceptable ingredient, including active agents and additives alike. Examples of such ingredients include other solvents, structuring agents such as waxes and polymers, hydrophobic (lipophilic) and hydrophilic thickeners or gelling agents, skin conditioning agents (humectants, exfoliants or emollients), dispersion enhancing agents, fillers (e.g., powders and mother-of-pearl), fibers, sunscreen agents (e.g., octocrylene, octinoxate, avobenzone), preservatives (e.g., sodium citrate, phenoxyethanol, parabens and mixtures thereof), chelators (such as EDTA and salts thereof, particularly sodium and potassium salts), antioxidants (e.g., BHT, tocopherol), neutralizing or pH-adjusting agents (e.g., sodium hydroxide), and cosmetically active agents and dermatological active agents such as, for example, additional skin care actives such as peptides (e.g., Matrixyl [pentapeptide derivative]), farnesol, bisabolol, phytantriol, vitamins and derivatives thereof such as ascorbic acid, vitamin A (e.g., retinoid derivatives such as retinyl palmitate or retinyl propionate), vitamin E (e.g., tocopherol acetate), vitamin B3 (e.g., niacinamide) and vitamin B5 (e.g., panthenol), anti-acne agents (resorcinol and salicylic acid), antioxidants (e.g., phytosterols and lipoic acid), flavonoids (e.g., isoflavones, phytoestrogens), and agents suitable for aesthetic purposes such as essential oils, fragrances, skin sensates, opacifiers, aromatic compounds (e.g., clove oil, menthol, camphor, eucalyptus oil, and eugenol), anti-inflammatory agents, defoaming agents, and essential fatty acids.

The cosmetic compositions of the present disclosure may also contain a wax. Examples of waxes of animal origin include beeswaxes, lanolin waxes and Chinese insect waxes. Examples of waxes of plant origin include rice waxes, carnauba wax, candellila wax, ouricurry wax, cork fibre waxes, sugar cane waxes, Japan waxes, sumach wax and cotton wax. Examples of waxes of mineral origin include paraffins, microcrystalline waxes, montan waxes and ozokerites. Examples of waxes of synthetic origin include polyolefin waxes, e.g., polyethylene waxes, waxes obtained by Fischer-Tropsch synthesis, waxy copolymers and their esters, and silicone and fluoro waxes. Alternatively, hydrogenated oils of animal or plant origin may be used. Examples include hydrogenated jojoba waxes and hydrogenated oils which are obtained by catalytic hydrogenation of fats composed of a C8-C32 linear or nonlinear fatty chain, hydrogenated sunflower oil, hydrogenated castor oil, hydrogenated copra oil, hydrogenated lanolin and hydrogenated palm oils. The wax may be present in the compositions in an amount generally ranging from about 0% to about 50%, based on the total weight of the composition.

In another embodiment, provided herein are patches comprising one or more peptides having an amino acid sequence selected from the group consisting of KGGRAKD (SEQ ID NO:1), KGG, GGR, GRA, RAK, AKD, DKA, KAR, ARG, RGG, GGK, DKARGGK (SEQ ID NO:2), ACTGSTQHQGGGSEHGAMEI (SEQ ID NO: 3), ACSSSPSKHCGGGSESPLKRQG (SEQ ID NO: 4), and ACTGSTQHQGGGSESPLKRQG (SEQ ID NO: 5) or a variant or mimetic thereof. The patch may include an adhesive skin patch, a face mask, a microneedle patch, and a hyaluronic acid patch, etc. The patch may include a front side which is to be applied on the surface of the skin, and where a cosmetic composition is provided, and a rear side. The microneedles in the microneedle patch may be classified as solid microneedles for the pretreatment of skin, coated microneedles with water-soluble formulations, dissolving microneedles without residual fragments, and hollow microneedles for liquid formulations. The microneedles may also include a disposable-manner microneedle made of carboxy-methyl-cellulose, a multi-round responsive microneedle made of alginate, a temperature responsive microneedle made of vinyl pyrrolidone, a glucose responsive microneedle made of hyaluronic acid, a pH responsive microneedle made of hyaluronic acid, a swelling-shrinking microneedle made of hydrogel, a water-soluble microneedle made of dextrin, etc.

In some embodiments, provided herein are methods for improving skin rejuvenation. The improving skin rejuvenation may include anti-aging, detoxification, anti-glycation, collagen regeneration, and/or improving elasticity and/or youthfulness of skin. The skin elasticity may be the skin's ability to stretch and snap back to its original shape. For instance, the skin elasticity may be measured based on a suction method where a negative pressure is produced in the measuring head, and the skin is drawn inside the instrument. An optical measuring system consisting of a light source and light receptor measures the light intensity, which varies in accordance with the degree of skin penetration. The two parameters measured are firmness and elasticity. Firmness is measured in terms of the resistance that the skin displays against being drawn in by the negative pressure. Elasticity is measured in terms of the time taken for the skin to return to its original state.

Loss of skin elasticity is also known as elastosis which causes skin to look saggy, crinkled, or leathery. The areas of the skin exposed to the sun can get solar elastosis. Therefore, the cosmetic compositions of the present disclosure may be applied to the skin of a subject who needs improvement of elasticity and/or youthfulness of the skin. In another embodiment, the cosmetic compositions of the present disclosure may be applied to a subject suffering from elastosis. In some embodiments, the cosmetic compositions of the present disclosure may be applied to a subject who needs anti-aging treatment. In some embodiments, applying the cosmetic composition of the present disclosure to the skin of the subject may reduce a fructose level (or glucose, sucrose or other polysaccharides level) in adipocytes or skin cells in the subject. In another embodiment, applying the cosmetic composition to the skin of the subject may prevent collagen degradation in the subject.

Moreover, the patches of the present disclosure may be applied to the skin of a subject who needs improving skin rejuvenation such as anti-aging, detoxification, anti-glycation, collagen regeneration, and/or improving elasticity and/or youthfulness of skin. In some embodiments, applying the patches of the present disclosure to the skin of the subject may reduce a fructose level (or glucose, sucrose or other polysaccharides level) in adipocytes and/or skin cells in the subject. In another embodiment, applying the cosmetic composition of the present disclosure to the skin of the subject may prevent collagen degradation in the subject.

In certain embodiments, the peptides described herein (or variants thereof) are applied to the skin of the subject in an amount, expressed as a daily equivalent dose regardless of dosing frequency, of 1 micrograms (“mcg”) per day, 2 meg per day, 3 mcg per day, 4 mcg per day, 5 mcg per day, 6 meg per day, 7 meg per day, 8 meg per day, 9 meg per day, 10 mcg per day, 15 mcg per day, 20 mcg per day, 25 mcg per day, 30 mcg per day, 35 mcg per day, 40 mcg per day, 45 mcg per day, 50 mcg per day, 60 mcg per day, 70 mcg per day, 75 mcg per day, 100 mcg per day, 150 mcg per day, 200 mcg per day, or 250 mcg per day. In some embodiments, the peptides described herein (or variants thereof) are administered in an amount of 500 mcg per day, 750 mcg per day, or 1 milligram (“mg”) per day. In another embodiments, the daily equivalent dose may be in the range of 1 mcg per day to 1 mg per day. The upper and lower limits may be alternatively selected in any values in the above range.

In yet further embodiments, the peptides described herein are administered in an amount, expressed as a daily equivalent dose regardless of dosing frequency, of 1-10 mg per day, including 1 mg per day, 1.5 mg per day, 1.75 mg per day, 2 mg per day, 2.5 mg per day, 3 mg per day, 3.5 mg per day, 4 mg per day, 4.5 mg per day, 5 mg per day, 5.5 mg per day, 6 mg per day, 6.5 mg per day, 7 mg per day, 7.5 mg per day, 8 mg per day, 8.5 mg per day, 9 mg per day, 9.5 mg per day, or 10 mg per day. In another embodiments, the daily equivalent dose may be in the range of 1 mg per day to 10 mg per day. The upper and lower limits may be alternatively selected in any values in the above range.

In various embodiments, the peptides described herein (or variants thereof) are applied to the skin of the subject on a monthly, biweekly, weekly, daily (“QD”), or twice a day (“BID”) dosage schedule. In other embodiments, the peptide/polypeptide is applied to the skin of the subject. In typical embodiments, the peptide/polypeptide is administered transdermally for at least 3 months, at least 6 months, at least 12 months, or more. In some embodiments, peptides described herein (or variants thereof) are administered transdermally for at least 18 months, 2 years, 3 years, or more.

### EXAMPLES

### Example 1

### Example 1-1

Fructose concentration in 3T3-L1 cell lysates was measured using the Fructose Assay Kit (Abcam, Cambridge, UK). Six-well plates were seeded with 5×10⁵ 3T3-L1 cells/well one day before treating with 150 μM peptide. After 48 hours of treatment, the cells were harvested and fructose concentrations were assayed according to the manufacturer's instruction.

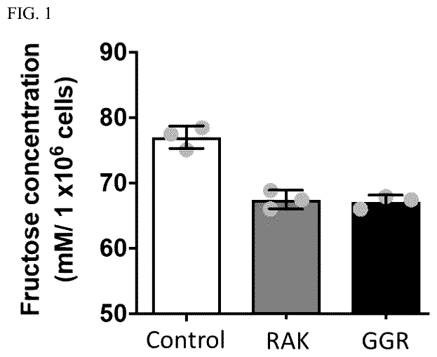

In order to confirm the effect of peptides on reducing the fructose level in adipocytes, three groups of adipocytes were prepared. Among the three groups, one group was treated with GGR, and another group was treated with RAK. The remaining group was not treated with any peptides and used as a control group. Then, the fructose concentration in each group was measured, and the results are demonstrated in FIG. 1. Fructose—an intermediate metabolite of glucose—was measured to further probe glucose metabolism. 3T3-L1 cells treated with either peptide had lower fructose concentrations than untreated cells (FIG. 1).

As shown in FIG. 1, the fructose concentrations in the groups where GGR and RAK were treated were significantly lower than the control group.

Therefore, it was confirmed that GGR or RAK reduces the fructose level in adipocytes.

### Example 1-2

An additional group of adipocytes is treated with KGGRAKD in the same manner as Example 1-1.

The fructose concentration in the group where KGGRAKD is treated is significantly lower than the control group.

Therefore, KGGRAKD reduces the fructose level in adipocytes.

### Example 1-3

An additional group of adipocytes is treated with KGG in the same manner as Example 1-1.

The fructose concentration in the group where KGG is treated is significantly lower than the control group.

Therefore, KGG reduces the fructose level in adipocytes.

### Example 1-4

An additional group of adipocytes is treated with GRA in the same manner as Example 1-1.

The fructose concentration in the group where GRA is treated is significantly lower than the control group.

Therefore, GRA reduces the fructose level in adipocytes.

### Example 1-5

An additional group of adipocytes is treated with AKD in the same manner as Example 1-1.

The fructose concentration in the group where AKD is treated is significantly lower than the control group.

Therefore, AKD reduces the fructose level in adipocytes.

### Example 1-6

An additional group of adipocytes is treated with DKA in the same manner as Example 1-1.

The fructose concentration in the group where DKA is treated is significantly lower than the control group.

Therefore, DKA reduces the fructose level in adipocytes.

### Example 1-7

An additional group of adipocytes is treated with KAR in the same manner as Example 1-1.

The fructose concentration in the group where KAR is treated is significantly lower than the control group.

Therefore, KAR reduces the fructose level in adipocytes.

### Example 1-8

An additional group of adipocytes is treated with RGG in the same manner as Example 1-1.

The fructose concentration in the group where RGG is treated is significantly lower than the control group.

Therefore, RGG reduces the fructose level in adipocytes.

### Example 1-9

An additional group of adipocytes is treated with ARG in the same manner as Example 1-1.

The fructose concentration in the group where ARG is treated is significantly lower than the control group.

Therefore, ARG reduces the fructose level in adipocytes.

### Example 1-10

An additional group of adipocytes is treated with GGK in the same manner as Example 1-1.

The fructose concentration in the group where GGK is treated is significantly lower than the control group.

Therefore, GGK reduces the fructose level in adipocytes.

### Example 1-11

An additional group of adipocytes is treated with DKARGGK in the same manner as Example 1-1.

The fructose concentration in the group where DKARGGK is treated is significantly lower than the control group.

Therefore, DKARGGK reduces the fructose level in adipocytes.

### Example 1-12

An additional group of adipocytes are treated with one of KGGRAKD, KGG, GGR, GRA, RAK, AKD, DKA, KAR, ARG, RGG, GGK, and DKARGGK in the same manner as Example 1-1.

The glucose concentration in the groups where the above peptides are treated is significantly lower than the control group.

Therefore, the above peptides reduce the glucose level in adipocytes.

### Example 1-13

An additional group of adipocytes are treated with one of KGGRAKD, KGG, GGR, GRA, RAK, AKD, DKA, KAR, ARG, RGG, GGK, and DKARGGK in the same manner as Example 1-1.

The sucrose concentration in the groups where the above peptides are treated is significantly lower than the control group.

Therefore, the above peptides reduce the sucrose level in adipocytes.

### Example 1-14

An additional group of skin cells are treated with one of KGGRAKD, KGG, GGR, GRA, RAK, AKD, DKA, KAR, ARG, RGG, GGK, and DKARGGK in the same manner as Example 1-1.

The fructose concentration in the groups where the above peptides are treated is significantly lower than the control group.

Therefore, the above peptides reduce the fructose level in skin cells.

### Example 1-15

An additional group of skin cells are treated with one of KGGRAKD, KGG, GGR, GRA, RAK, AKD, DKA, KAR, ARG, RGG, GGK, and DKARGGK in the same manner as Example 1-1.

The glucose concentration in the groups where the above peptides are treated is significantly lower than the control group.

Therefore, the above peptides reduce the glucose level in skin cells.

### Example 1-16

An additional group of skin cells are treated with one of KGGRAKD, KGG, GGR, GRA, RAK, AKD, DKA, KAR, ARG, RGG, GGK, and DKARGGK in the same manner as Example 1-1.

The sucrose concentration in the groups where the above peptides are treated is significantly lower than the control group.

Therefore, the above peptides reduce the sucrose level in skin cells.

### Example 1-17

An additional group of skin cells were treated with ACTGSTQHQGGGSESPLKRQG (SEQ ID NO: 5) in the same manner as Example 1-1.

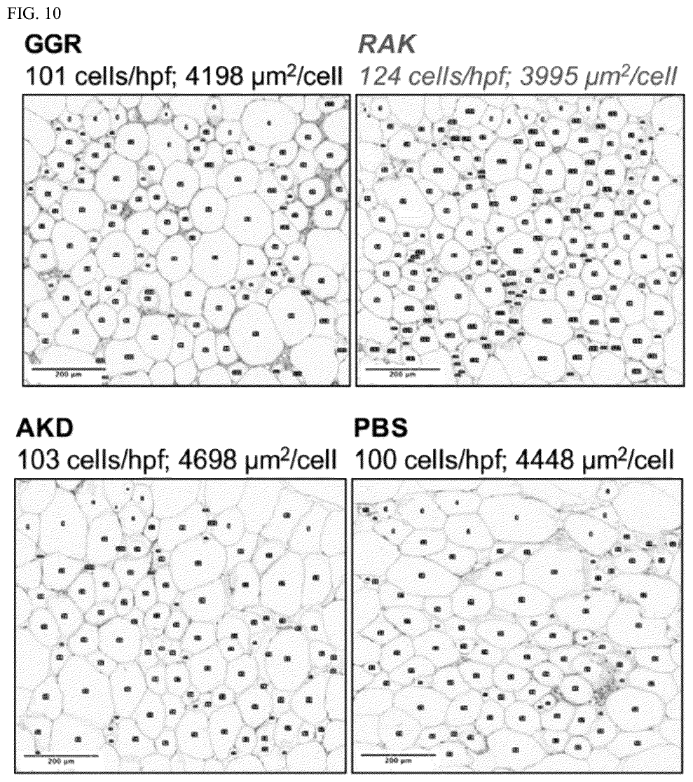

The fructose, glucose, and sucrose concentrations in the groups where the above peptide was treated were not affected (data not shown), while the extra cellular matrix molecules, collagen and fibronectin, were determined. The collagen and fibronectin in the group where ACTGSTQHQGGGSESPLKRQG (SEQ ID NO: 5) was treated were significantly higher than the control group at a particular concentration. See FIG. 11. Therefore, the above peptide reduced the sucrose level in skin cells, but improved the production of collagen and fibronectin.

### Example 1-18

An additional group of skin cells are treated with one of ACTGSTQHQGGGSEHGAMEI (SEQ ID NO: 3) and ACSSSPSKHCGGGSESPLKRQG (SEQ ID NO: 4) in the same manner as Example 1-1.

The fructose, glucose, and sucrose concentrations in the groups where the above peptides are treated are not affected (data not shown), while the extra cellular matrix molecules, collagen and fibronectin, are determined. The collagen and fibronectin in the group where ACTGSTQHQGGGSEHGAMEI (SEQ ID NO: 3) or ACSSSPSKHCGGGSESPLKRQG (SEQ ID NO: 4) is treated are significantly higher than the control group at a particular concentration.

Therefore, the above peptides reduce the sucrose level in skin cells, but improve the production of collagen and fibronectin.

### Example 2

### Assay of Peptide Activity in In Vitro Human System

In murine-based in vitro systems, GGR, RAK, and AKD were effective in reducing lipid accumulation. These peptides are expected to play similar roles in human mature adipocytes in inhibiting lipid accumulation and adipogenesis. This example describes testing of lead peptide candidates, GGR, RAK, and AKD, in human adipocytes for improved translational potential. Due to structural similarities between murine and human physiology, interactions of the short peptides with human mature adipocytes are predicted to show similar results in reducing lipid accumulation that were seen in mouse adipocytes.

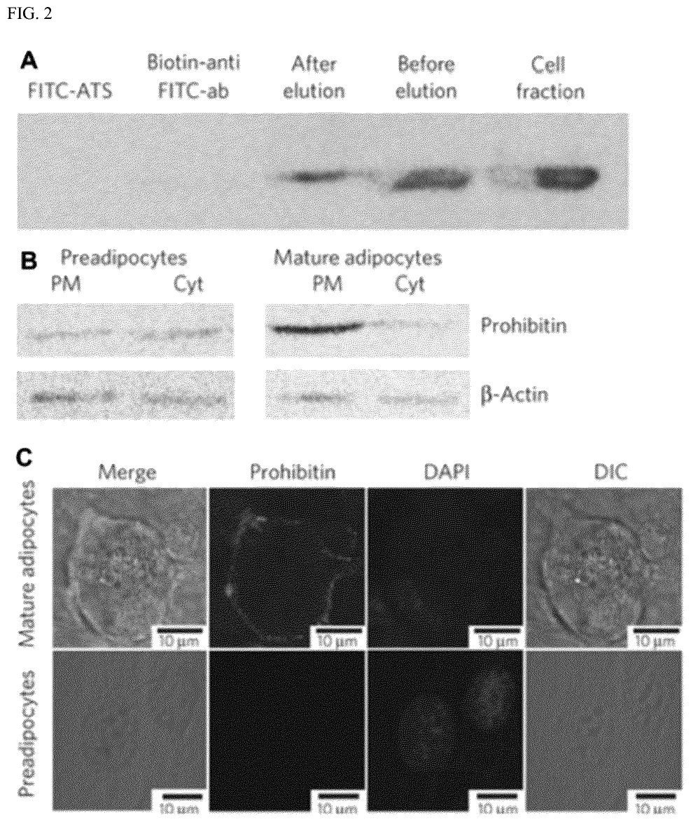

It was confirmed that prohibitin shifts its location to the cell membrane as adipogenesis progresses and ATS binds to prohibition. The ATS/prohibitin binding was verified in pre- or mature adipocytes (Won, Y. W. et al. Nat Mater 13, 1157-1164, (2014)). Fractionated proteins isolated from adipocytes were incubated with the ATS magnetic beads and the protein captured by ATS was identified by western blotting. The bound protein to ATS was revealed to be prohibitin (A of FIG. 2). This indicates that ATS specifically binds to prohibitin. Prohibitin is observed in several intracellular locations at varying expression levels depending on the degree of adipogenesis (Wang, P. et al. Cell Mol Life Sci 61, 2405-2417, (2004); Patel, N. et al. Proceedings of the National Academy of Sciences of the United States of America 107, 2503-2508, (2010)). The degree and location of prohibitin expression in pre- or mature adipocytes was determined. More prohibitin is found on the cell membrane (PM) of mature adipocytes, while no difference in prohibitin expression was observed in pre-adipocytes (B of FIG. 2). Confocal microscopy also verified the prohibitin expression on the cell membrane of mature adipocytes (C of FIG. 2). High-level prohibitin expression on the cell membrane of mature adipocytes is important to the ATS/prohibitin interaction.

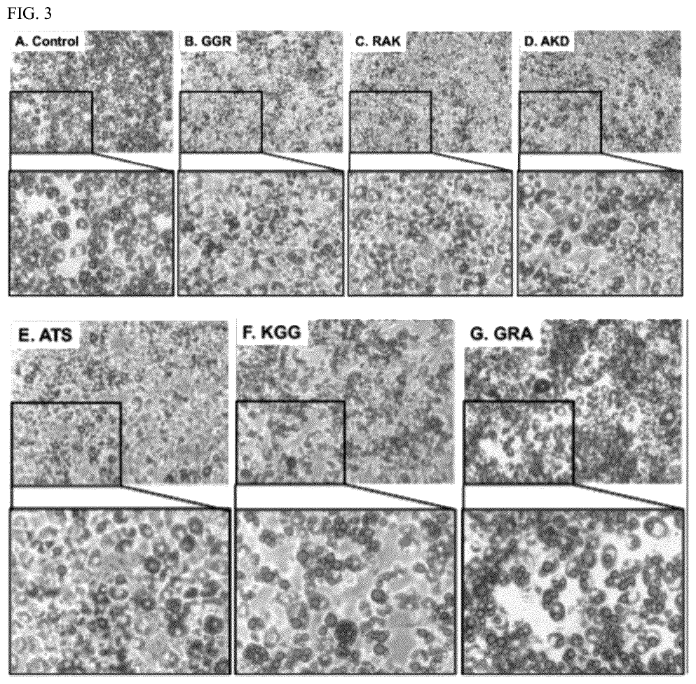

The effects of the minimal short peptides in blocking lipid accumulation in adipocytes were compared with their parent full-length peptide. After generating five different 3 amino acid fragments (KGG, GGR, GRA, RAK, and AKD) from the original ATS peptide (KGGRAKD (SEQ ID NO:1)), each peptide was administered to 3T3-L1 pre-adipocytes in five varying concentrations. Peptides were treated to the adipocytes three times per week for three weeks. Oil Red O staining was utilized to assess the lipid contents in mature adipocytes in culture. The morphological characteristics of 3T3-L1 mature adipocytes were observed by microscopy. The number and size of lipid droplets in adipocytes treated with GGR, RAK, AKD, or ATS (B-E of FIG. 3) were smaller than those in the non-treated control (A of FIG. 3), whereas moderate differences in the morphology of lipid droplets were observed between KGG and GRA (F, G of FIG. 3) vs. control. Furthermore, the lipids were quantitatively analyzed. This result demonstrates that the degree of lipid accumulation in 3T3-L1 mature adipocytes was decreased by approximately 20% upon treatment of GGR, RAK, or AKD (H of FIG. 3). At the end point, the cell number was almost the same across the treatment groups, meaning that the reduction in lipid accumulation is not a consequence of toxicity of the peptides (I of FIG. 3). The most effective peptide candidates were screened. These results identified GGR, RAK, and AKD as lead peptides.

Effects of GGR, RAK, and AKD on lipid accumulation are validated in human adipocytes. Primary human subcutaneous pre-adipocytes are purchased from American Type Culture Collection (ATCC) to be used in all in vitro studies (ATCC PCS-210-010). The adipocyte differentiation-inducing described above is optimized for the generation of human mature adipocytes from the human pre-adipocytes (Zebisch, K., et al., Anal Biochem 425, 88-90, (2012)). Naive human pre-adipocytes serve as the undifferentiated control. The cells are first thawed into two T75 flasks per 1 million cell vial in basal medium I (BMI), which is composed of DMEM (4.5 g/l glucose) supplemented with 10% FBS and 1% Penicillin Streptomycin (P/S). The media is changed the following day, and on day 3, the cells are seeded in 96-well plates (200 μl/well). On day 4, the media is changed to DMI, which is DMEM containing 10% FBS, 1% P/S, 1 μg/μl insulin, 0.5 mM IBMX, and 0.25 UM Dexamethasone. By this day, the viscosity of the media will have increased due to lipid production by the cells. On day 7, the media is changed to DMII, which is DMEM (4.5 g/l glucose) supplemented with 10% FBS, 1% P/S, and 1 μg/μl insulin. It is expected that intracellular droplets are seen after one week of culture and adipogenesis is observed. At the time of full differentiation, the media is changed back to BMI. For the control pre-adipocytes, the media is replenished on days 3, 5, 7, 8, and 12 (Zebisch et al., supra).

The testing of various peptide concentrations accommodates a dose-dependent effect on differentiation and determines optimal doses for use in in vivo studies. To verify the anti-obese effects of the three peptides in human systems, the same protocol as devised for the in vitro animal cell-based systems is used. Cells are maintained in the BMI culture media until the peptide treatments begin (days 12-14). Five different fragments (KGG, GGR, GRA, RAK, and AKD) and the complete ATS sequence (KGGRAKD (SEQ ID NO:1)) are tested on the adipocytes. Various peptide concentrations are used to elucidate the relationship of dose and efficacy of peptide treatment. A monoclonal antibody (mAb) against prohibitin is used as a positive control (Invitrogen, MA5-12858).

The number of viable cells is counted prior to Oil Red O staining using Cell Counting Kit 8 (Dojindo). This cell viability study allows one to determine potential toxicities of the peptides and compensate for cell number variation-causing artifacts. Oil Red O is dissolved in isopropanol at 3 μg/ml to create a working solution. 12 ml of Oil Red O working solution is mixed with 8 ml dH₂O and incubated for 10 minutes. This solution is then filtered using a 0.2 μm syringe filter. Adipocytes seeded on 96-well plates are washed with PBS twice. 10% formaldehyde is added for initial fixation and the plates are incubated for at least 30 minutes. The formaldehyde is removed and the cells are once again washed twice, this time with dH₂O. 60% isopropanol is added to each well. The plates are incubated for 5 minutes. The isopropanol is removed from the wells and replaced with Oil Red O working solution. The plates are incubated for an additional 10 minutes. The Oil Red O solution is removed and washed four times with dH₂O. Lastly, digital micrographs of the lipid droplets in adipocytes are taken using a light microscope. After the staining protocol, all plates are washed three times with 60% isopropanol. Each time, 5 minutes of gentle rocking was used. The Oil Red O is extracted with 100% isopropanol with gentle rocking for 5 minutes. 200 μl isopropanol per well is used as a background to measure absorbance at 492 nm using a BioTek microplate reader.

Effects of ACTGSTQHQGGGSESPLKRQG (SEQ ID NO: 5) on collagen and fibronectin production are validated in mouse fibroblast. A mouse fibroblast cell line is purchased from American Type Culture Collection (ATCC) to be used in all in vitro studies (3T3-L1 CL-173). The cells are first thawed into two T75 flasks per 1 million cell vial in basal medium I (BMI), which is composed of DMEM (4.5 g/l glucose) supplemented with 10% FBS and 1% Penicillin Streptomycin (P/S). The media is changed the following day, and on day 3, the cells are seeded in 96-well plates (200 μl/well). The peptide was administered to 3T3-L1 fibroblast in five varying concentrations. Collagen and fibronectin produced from the cell was assessed in the cell culture media 3 days after the peptide treatment by ELISA. At the 0.2 uM concentration, both collagen and fibronectin levels were increased compared to the non-treated control, whereas the levels gradually decrease as the peptide concentration increases. This result demonstrates that the production of extra-cellular matrix molecules in 3T3-L1 fibroblast was increased by the peptide treatment. At the end point, the cell number was almost the same across the treatment groups, meaning that the peptide is non-toxic. The most effective peptide candidates were screened. These results identified ACTGSTQHQGGGSESPLKRQG (SEQ ID NO: 5) as the lead peptide.

### Example 3

### Efficacy of the Peptides in Animal Models

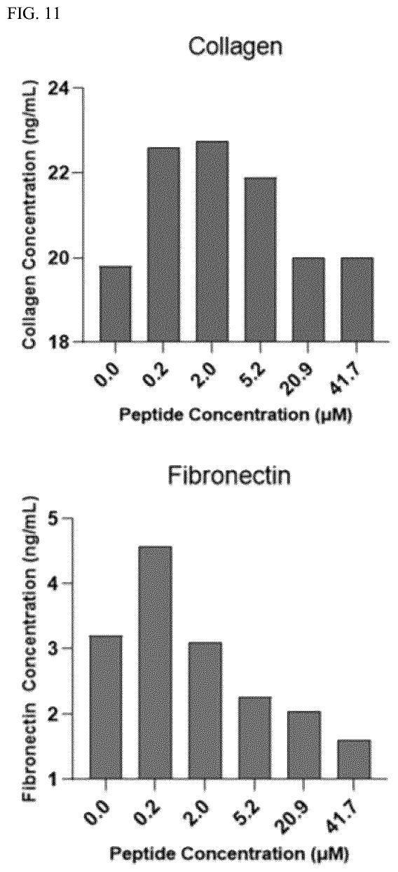

Prior to oral administration, peptides were administered to DIO mice via intraperitoneal (IP) and subcutaneous (SC) injections. This study was divided into two phases as shown in FIG. 12.

In the first phase, peptides were tested on pre-obese mice that were being fed high fat diet (HFD) to induce obesity (obesity progression model, OPM). In the second phase, these peptides were given to mice that had already developed obesity (obesity model, OM). GGR and RAK exhibited positive effects on prevention of body weight gain in both OPM and OM mice given IP injections. Scrambled peptide, RKG, did not show meaningful efficacy over the vehicle group (PBS). With SC injections, GGR and RAK were effective in OM mice, while AKD turned out to be the best working peptide in OPM mice. As the activity of these three peptides varies depending on the injection route and the disease status, all three peptides, GGR, RAK, and AKD, were tested by oral administration. In OPM mice given oral administration, RAK and AKD were efficacious over GGR. In OM mice, RAK, GGR, and AKD led to similar levels of obesity prevention.

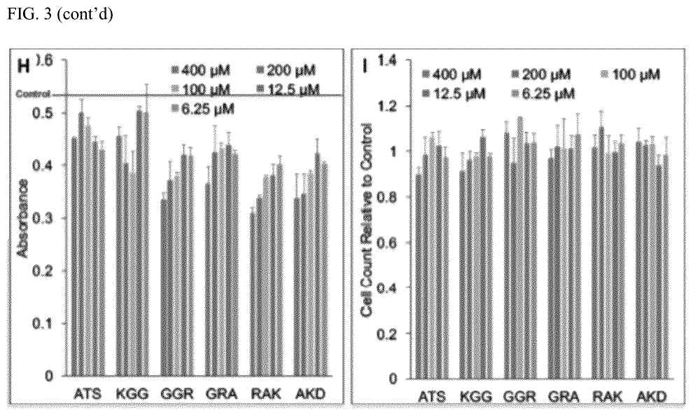

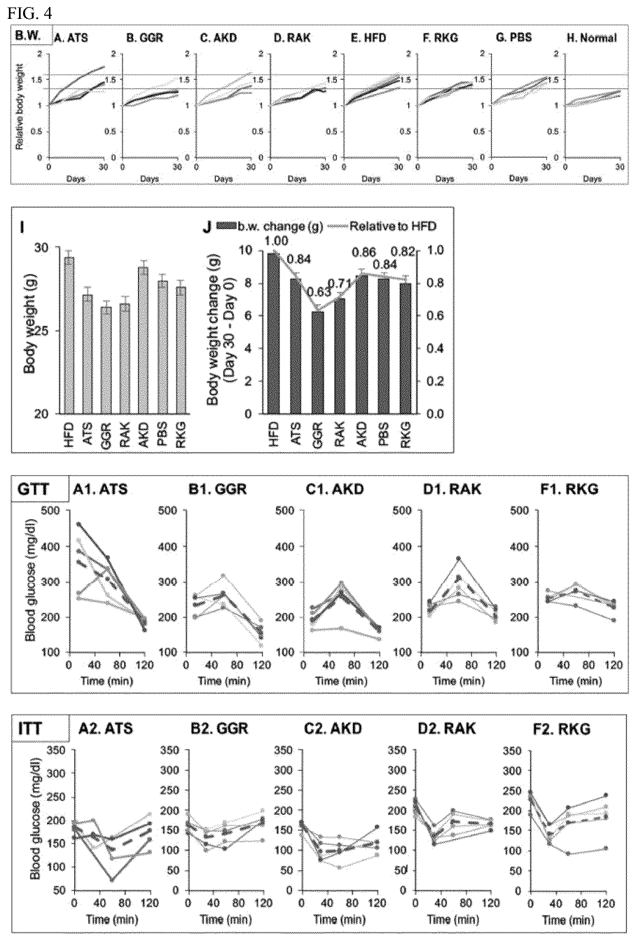

Prior to oral administration, GGR, RAK, AKD, full-length ATS, and scrambled RKG peptides were administered to OPM mice via IP and SC injections to further screen an ideal peptide composition. PBS was included as a vehicle group. GGR, RAK, and AKD peptides were discovered to impede body weight gain in OPM mice. GGR and RAK were effective when given IP injections and AKD was effective by SC injections (FIGS. 4 and 5). Scrambled RKG and vehicle showed no anti-obese effects.

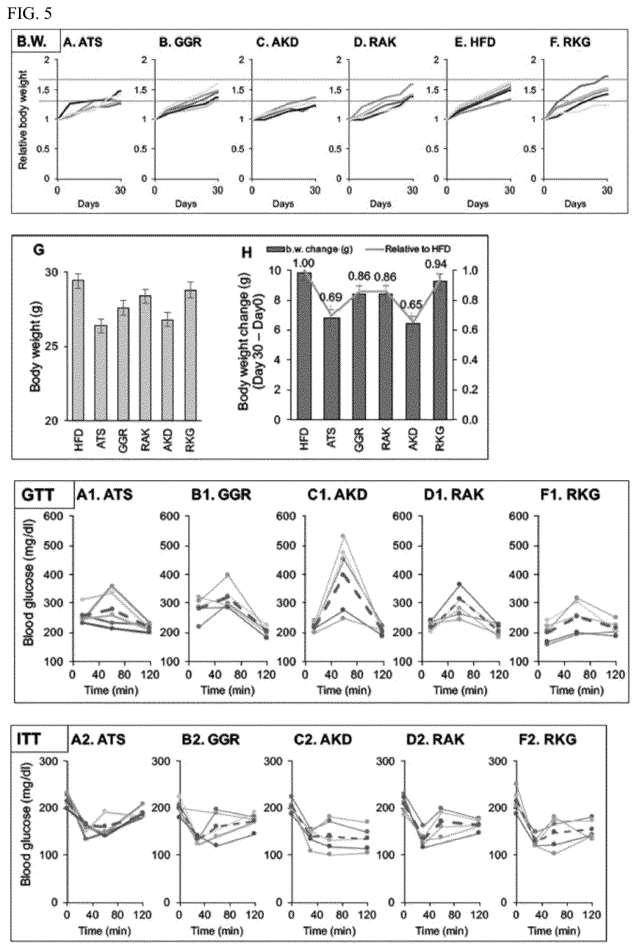

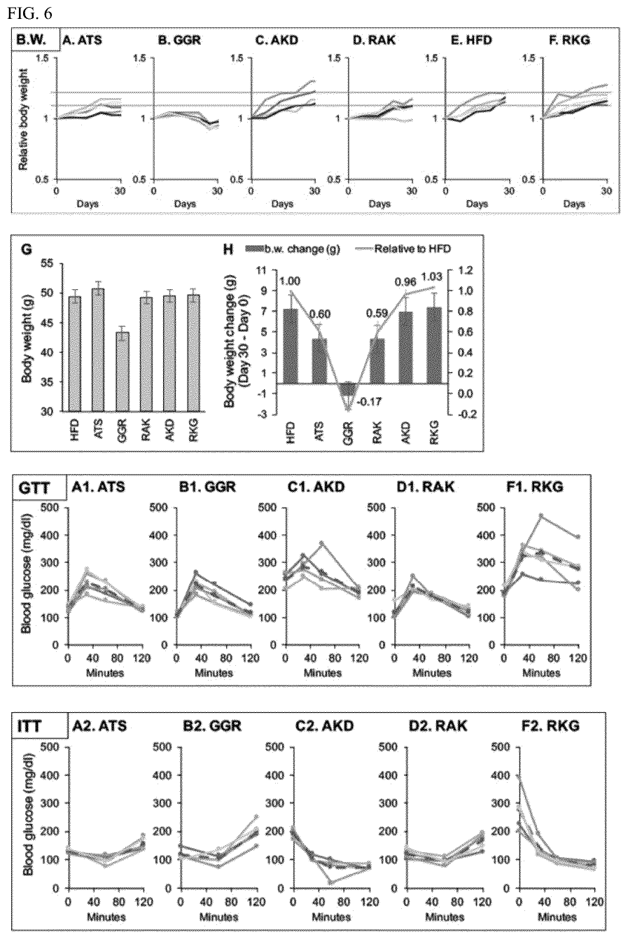

The second phase signified the post-obesity stage, where the same methods from phase 1 were used on OM mice. Body weight change showed that GGR and RAK exhibited similar activity to that of the parent ATS peptide in both injection routes in post-obese mice (FIGS. 6 and 7). These results further confirm that GGR and RAK were the most promising peptide compositions.

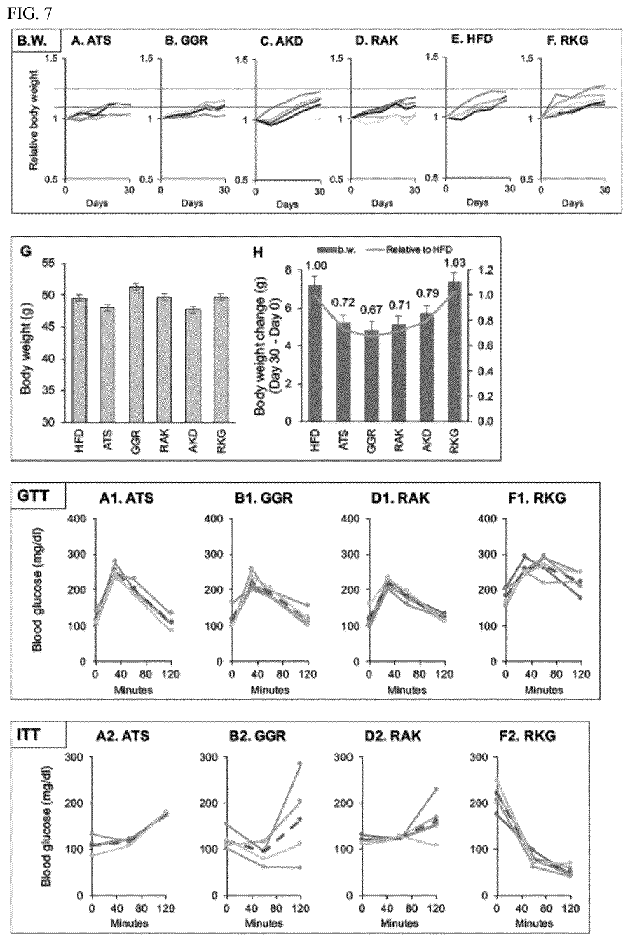

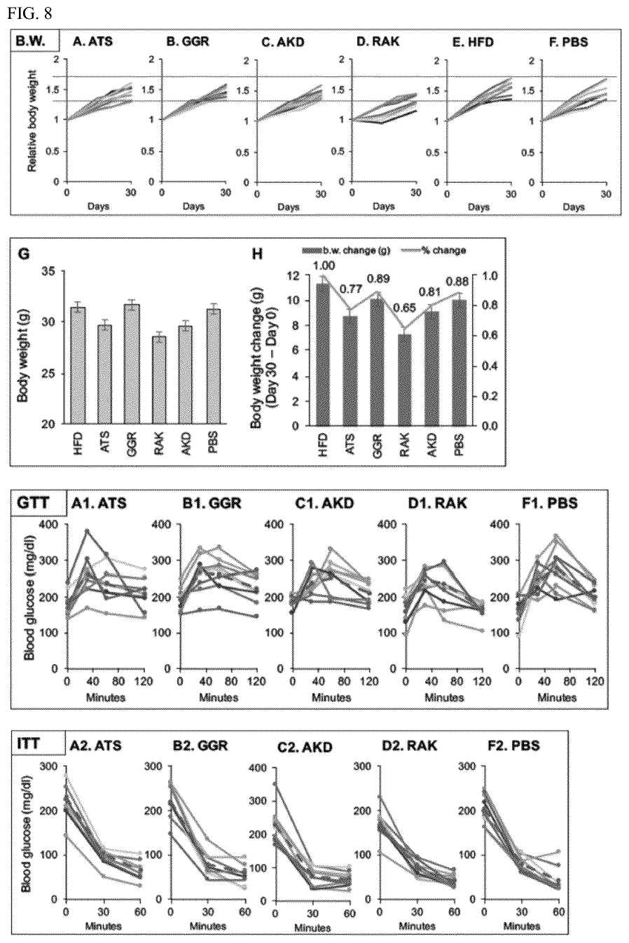

Through the IP and SC studies, it was found that scrambled RKG peptide and vehicle (PBS) had no anti-obesity effects regardless of injection route or schedule. GGR, RAK, and AKD peptides were tested in OPM and OM mice by oral administration. FIGS. 8 and 9 demonstrate that RAK has anti-obesity efficacy in both models and GGR should be further validated. Histological analysis of the fat pads verifies that RAK is more active than GGR. In obesity progression, adipocytes increase in both number and size due to escalating lipid accumulation (Parlee, S. D., et al., Methods Enzymol 537, 93-122, (2014)). Using the methods described above, abdominal fat pads of GGR, RAK, AKD, or PBS-treated DIO mice were histologically analyzed. Epididymal fat tissues were harvested from two mice per experimental group. All samples were fixed in 4% paraformaldehyde, H&E-stained, and imaged. At a 10× magnification, adipocyte cell counting was performed on the digital slide images using an automated adipocyte counting macro with the ImageJ software. A default thresholding method was used and [cell] sizing boundaries of 40-40,000 were set. Limitations of the segmentation tool led to some erroneous selections or regions of interest (ROI), which were manually corrected by adding or erasing lines. The number of cells per representative field were then determined using the ROI manager in ImageJ. Two or three high power fields (hpf) per tissue and two tissue samples per group were analyzed to reduce variability. Average cell count for each group was calculated accordingly. Areas of all adipocytes of interest were measured using the ROI measurement tool (ImageJ) and average area per cell was calculated accordingly.

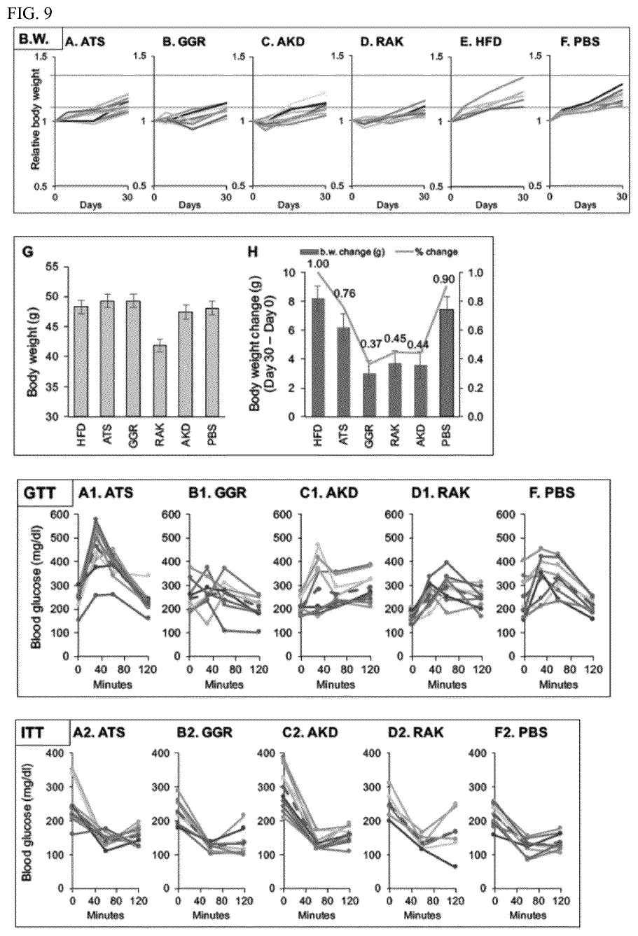

The RAK group was found to have the highest average cell count per field resulting in the lowest average adipocyte area per cell among all experimental groups (FIG. 10). The primary function of adipocytes is to store energy in the form of lipids. The volume of lipid stored in an adipocyte cell increases as the third power of the diameter. Because mature adipocytes are composed of large lipid droplets that are surrounded by very thin cytoplasmic layers, the increase in volume of an adipocyte cell likely indicates increased lipid storage. Thus, smaller adipocyte size/area translates to reduced lipid accumulation. The results show that RAK demonstrates the highest efficacy in this context.

Genetic or dietary models are well-established preclinical models of obesity being used in researches and drug discoveries (Barrett, P., et al., Dis Model Mech 9, 1245-1255, (2016)). Genetic models, including spontaneous mutant and transgenic animals, have been used to define the mechanism of obesity progression (Zhang, Y. et al. Nature 372, 425-432, (1994); Huszar, D. et al. Cell 88, 131-141 (1997)). Since multiple genes contribute to the development of human obesity, obesity is considered polygenic rather than monogenic in nature. Traditional genetic models are now considered as useful mouse models to understand obesity because these monogenic models do not reflect the diversity of human diseases. Dietary models have been advanced to develop polygenic DIO models that better mimic the complex process of human obesity. A DIO model needs fine-tuning, such as dietary choices and meal-type feeding/drinking regimes, to better simulate the heterogeneity of human obesity, an aspect that is not easily mimicked in experimental animals. Recent advances in the development of pre-clinical models of obesity support the use of models that represent the outcome of gene/environment interaction in order to minimize potential artifacts caused by behavioral changes (Barrett et al., supra). The peptides are investigated in 3 pre-clinical models of obesity with 2-3 conditions.

The peptides are further validated in preclinical DIO models. Recent studies have shown the advances in DIO models and the advantages in using them over genetic models (Barrett et al., supra). A DIO model in which there is a defined (high-fat, 60%) diet administered to the mice is used. Some models use variations in fat and sugar contents to assess dietary preference and temporal traits of individual mice to determine the outcomes of interventions such as those described below.

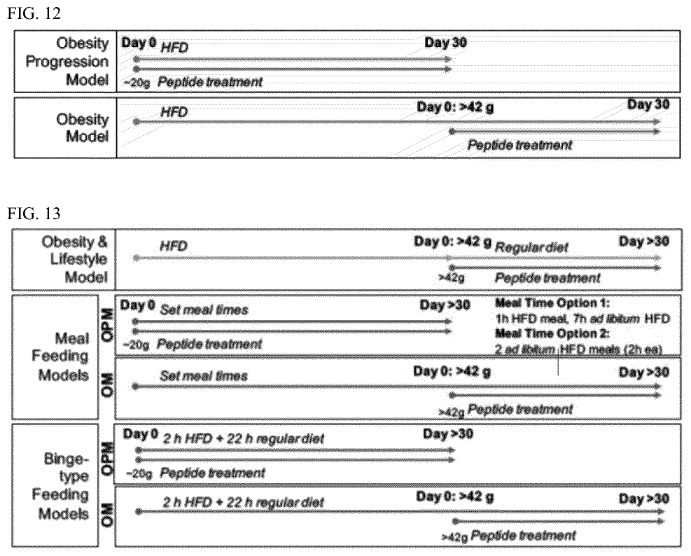

A DIO model generation scheme and injection schedule is shown in FIG. 13. Obesity with lifestyle change model: Continuous feeding of HFD may be too extreme and does not accurately reflect the life-style changes of a patient with obesity. Obese mice are generated via HFD; once obese, the HFD is changed to a regular diet (Tekland NIH-07) and treatment begins.

Meal-feeding model: Having unlimited (ad libitum) access to a high-energy diet does not translate well to human feeding behavior. Thus, an alternative model is providing set meals at specific times, which more closely imitates human dietary intake. A meal-feeding regime based on past studies and test is used to demonstrate HFD-induced weight gain most accurately.

Binge-type feeding model: Another human feeding behavior to consider is binge eating, which is characterized by overconsumption and lack of control. In a binge-type feeding intervention, animals have ad libitum access to HFD for 2 hours and regular diet for the remaining 22 hours of the day. This model is expected to increase body weight and fat mass in animals, having especially pronounced effects on C57BL/6 mice29. This model is used to observe the effects of extreme meal feeding on obesity progression and to assess the efficacy of the peptides in this particular context of weight gain.

Behavioral phenotyping is performed in DIO models. It is important to assess behavioral phenotypes in obesity models due to increasing evidence that early-life nutritional experiences and epigenetic gene regulation can influence diet choice later in life (Barrett et al., supra; Brenseke, B. et al. Endocrinology 156, 182-192, (2015)). With careful monitoring of specific dietary intake across time periods, it is possible to see which diets are preferred and when, given that the mice have a choice. In some embodiments, computerized systems such as the Comprehensive Lab Animal Monitoring System (Columbus Instruments) or PhenoMaster (TSE Systems) are used to measure food consumption using minute-by minute intervals. With such feeding data, it is possible to gather more information about DIO mice in terms of dietary patterns and preferences and the reasons for variations that we may observe. For example, exposure of obesity-inducing diets to otherwise normal mice may lead to an unpredictable course of weight gain, thus indicating susceptibility or resistance to DIO. Because obesity is a polygenic disease, different diets and feeding regimes are utilized to manipulate genetic models and in turn study environmental and genetic influences on obesity (Barrett et al., supra).

Metabolic changes are monitored. A Micro-Oxymax system (Columbus Instruments) is used for indirect calorimetric (IC) measurements (Matoba, K. et al. Cell Rep 21, 3129-3140, (2017)). Metabolic chambers are used to measure VO2 and VCO2 in individual mice. Respiratory exchange ratio (RER) is calculated to quantify energy expenditure. All data is analyzed using 2-way ANOVA.

Three-week old male C57BL/6J mice are purchased from Jackson Labs. The negative control group is assigned to mice on a normal, low-fat diet (Tekland NIH-07) with no treatment. The positive control group is mice that are fed HFD (DIO Tekland Rodent Diet TD.06414) purchased from Envigo. A group of mice (n=5) on a normal diet are tested with peptide treatment to confirm that the peptides mechanism of action is not relevant in normal mice. The vehicle control group is fed the HFD and treated solely with the vehicle (PBS). Scrambled RKG is omitted because this peptide has no effect over PBS. The DIO treatment cohorts includes: 1) GGR and 2) RAK. Prohibitin mAb is used as a positive control in response to these cohorts (Invitrogen, MA5-12858). A sample size of 10 mice per group is be used unless specified otherwise. This experiment is repeated up to 3 times, hence a total of 2,400 mice—5 models (2 models require both OPM and OM)×10 mice×8 groups×3 repeats×2 dosages are needed to complete the study. Female mice are added to the study to account for possible gender-specific differences and effects in the mouse population in obese conditions.

Peptide formulations are made at concentrations of 60 μM. The mice are orally treated at a dosage of 0.01 mg/g/day or 0.02 mg/g/day three times per week. The dose is calculated specifically for each peptide according to their molecular weights (GGR=288 g/mol, RAK=374 g/mol). Due to the challenge of developing oral delivery systems for mAbs, which are prone to enzymatic degradation and unfolding in the GI tract, a parenteral injection route (subcutaneous) is used to administer prohibitin mAb (Awwad, S. & Angkawinitwong, U. Pharmaceutics 10, (2018)). The dose is calculated according to the mAb molecular weight. HFD feeding and treatment to OPM mice is initiated when their body weights reach approximately 20 g. For OM mice, DIO mice are assigned to the treatment group when their body weights approximately reach 42 g. Oral feeding is facilitated with the use of animal feeding needles (Fisher Scientific) on 1-ml BD syringes. Body weight is measured weekly for a longer term. Real-time live imaging to measure abdominal fat mass provides direct evidence for body weight loss as a consequence of reduced fat. Adipocyte tissue masses are assessed by Micro-CT using a Siemens Inveon Micro-CT scanner. Epididymal fat tissues are imaged on the Micro-CT scanner using CT contrast agents. By using this system and its built-in analytic software, it is possible to calculate fat masses (Wu, C. et al. J Clin Invest 127, 4118-4123, (2017)).

Glucose and insulin tolerance tests are performed in order to characterize the metabolic phenotype of mice and to gain a better understanding of the pathogenesis of obesity (Nagy, C. & Einwallner, E. J Vis Exp, (2018)). This test is used to evaluate the ability to regulate glucose metabolism. Initial blood glucose levels are measured at 16 hours post-fasting using a Care Touch diabetes testing kit. Glucose (3 g/kg body weight) is injected intraperitoneally to each mouse. Blood samples are collected at specific time points: 0, 30, 60, and 120 minutes post-injection. ITT is conducted to monitor whole-body insulin action. GTT and ITT is not performed on the same day. Initial blood glucose levels are measured at 6 hours post-fasting. Insulin (0.75 U/kg body weight) is injected intraperitoneally. Blood samples are collected over time.

It is important to measure lipid profile and obesity-related proteins in blood because reduced lipid uptake by adipocytes may affect these factors. Plasma is separated from all samples and submitted to a medical diagnostic laboratory for plasma lipid profile measurements. Total cholesterol and triglyceride contents is analyzed. Adipokine levels are measured using the Proteome Profiler Mouse Adipokine Array Kit (R&D Systems, ARY103). This analysis detects the levels of obesity related proteins, such as adiponectin, leptin, and TNF-α, in individual samples. It is contemplated that DIO mice with no treatment will have low plasma concentrations of adiponectin and higher-than-normal adipokine levels because adipokines, except for adiponectin, are upregulated in obesity (Inadera, H. Int J Med Sci 5, 248-262 (2008)). Measurement of adipokine levels are useful to assess the degree of pathogenesis in the DIO mice. Cholesterol, triglyceride, and free fatty acid levels in blood may change upon treatment. Total cholesterol, free cholesterol, triglyceride and fatty acid contents, and phospholipid levels are measured via plasma or sera samples using colorimetric enzymatic assays. All assays are performed according to manufacturer instructions from Wako Diagnostics. Free cholesterol in serum is quantitatively determined using the Free Cholesterol E assay (Wako Diagnostics, 993-02501) while total cholesterol is measured with Cholesterol E assay (Wako, 999-02601). Fatty acid contents is measured using the HR Series NEFAHR (2) (Wako, 999-34691, 991-34891, 993-35191, 276-76491). Triglyceride levels are measured with the LType Triglyceride M Assay (Wako, 464-01601).

Immunohistochemistry, proteomics analysis, and biodistribution studies to confirm that the changes in adipocytes are due to peptide effects. Histological analysis of fat pads is performed according to the procedure detailed above. In this study, fat tissues from multiple sites and an increased number of mice are analyzed to statistically generalize prior findings. In addition, the peptide is fluorescently- or radio-labeled for organ distribution. A proteomics service is used to detect expression levels of proteins in adipose tissue samples extracted from mice post-treatment. A differential proteomic analysis allows for analysis of adipose tissue, a heterogeneous tissue with a composition spectrum that includes white and brown Adipocytes (Shields, K. J. & Wu, C. Methods Mol Biol 1788, 243-250, (2018)).

The therapeutic lead peptide is tested in prohibitin knockout (KO) mice to confirm that this peptide exerts its activity in the correct molecular target. It is expected that prohibitin KO mice do not respond to the peptide. To counter the knockout model, a transgenic mouse (Mito-Ob) that overexpresses prohibitin in adipocytes is also developed. It is contemplated that Mito-Ob mice develop obesity independent of diet and the therapeutic lead peptide interferes with obesity progression in Mito-Ob mice. The efficacy of the potential therapeutic lead peptide is compared with current therapies. Three recently commercialized drugs are tested on the DIO mouse models with the same two-phase oral administration schedule used for peptide treatments. Body weight assessments, metabolic profiles, and histological images are analyzed to compare the efficacy of the therapies to that of the peptides.

A one-way analysis of variance (ANOVA) with Dunnetts post hoc is used to compare multiple groups (various peptides/treatments and concentrations) with respect to the control cohorts. Peptide treatment effects on body weight change in DIO mice are analyzed using a repeated measures two way ANOVA with Tukeys post hoc test. All statistical analyses are performed using GraphPad Prism.

### Example 4

### Absorption, Distribution, Metabolism, Excretion (ADME), and Toxicity Properties of Peptides

In vivo absorption, distribution, metabolism, and excretion of the lead RAK peptide is determined in a mouse model. In vitro toxicity screening is also performed. In order to detect absorption, in vivo experiments by oral administration (gavage) as compared to an intravenous (i.v.) administration to calculate bioavailability (F=AUCp.o./AUCi.v.×100) are performed. Administration of the therapeutic lead peptide at 3 doses (0.1 mg/kg, 1 mg/kg, and 10 mg/kg) by oral gavage in C57BL/6J mice (male and female) is compared to an i.v. dose of 1 mg/kg (tail vein injection) in order to calculate the bioavailability. Terminal serum-draws takes place at times 1, 5, 10, 15, 30, 60, 120 and 240 minutes post-administration in separate animals (n=3 for each time point, n=144 males and n=144 females) and levels of the peptide are determined from the samples using HPLC-MS/MS analysis. Area under the curve (AUC) graphs are generated and used to calculate bioavailability (F). If serum levels are detectable at the 240-minute time point, further time points are measured. Vehicle and serum/plasma stabilities are also performed to determine the shelf-life stability of the peptide in solution at room temperature. Measurements over time (5, 15, 30 mins, 1, 2, 4, 6, 8, 12, 24, 48, and 72 hrs) are performed using HPLC-MS/MS.

The volume of distribution is calculated based on the following formula: Vd=dose given (mg)/plasma concentration (mg/L). This is based on a first order kinetics and extrapolation of the kinetic curve after i.v. and oral gavage dosing. Plasma concentrations from absorption studies above are used to determine the plasma concentration at time zero as an extrapolation during the kinetic phase.

In order to determine the clearance of the lead peptide, mouse metabolic cages are utilized to capture all urine after dosing with expectations that the majority of the parent compound and metabolites are be removed via the kidneys. Urine is analyzed for the parent compound and projected metabolites (individual amino acids) using HPLC-MS/MS. Detection for the glucuronidation of the peptide is also measured. Clearance is calculated using the following formula: CL=rate of elimination (mg/h)/plasma concentration (mg/L). The half-life of the peptide is calculated based on the following formula: t1/2=0.693 X Vd/CL. Feces are also collected if the parent peptide and metabolites are insufficient to extrapolate back to 95-99% of the initial dose. In previous studies, the majority (>99%) of peptide administered into the GI tract is absorbed and does not re-enter the hepatic portal system.

WinNonlinR (Pharsight Co., CA, USA) is used to quantify and simulate predictive PK/PD parameters as compared to observed values. The simulation PK/PD parameters are Tmax, Cmax, ke, t1/2 and AUC.

Initial metabolism and toxicology of the peptide is calculated through In Vitro ADMET Laboratories (IVAL) toxicity tests for hepatotoxicity screens, p450 inhibition, CYP induction, cytotoxicity, Ames activity, and hERG testing (Charles River). In addition, the lungs, heart, liver, kidneys, spleen and brain are removed and weighed to determine differences between peptide vs. vehicle-treated animals. Also, fat tissues are fixed and H&E stained for histopathology to compare peptide-treated animals to control and vehicle-treated animals for the 120- and 240-minute time points.

Design-ExpertR (Stat-Ease, Minn., MN) software is used to design experiments and to conduct the multivariate statistical analyses of the data. All data is analyzed using the statistical software packages, SigmaPlotR/SigmaStatR (JandelSci.). Control group=1, naive group (n=3), and 1 vehicle-only (n=3) administration injected group, two routes of administration, for 8 time-points (3 mice/group×2 genders×4 doses (3 peptide+1 vehicle)×2 routes of administration×8 time-points+3 naive=387 mice in total). Experimenters are blinded to treatment and control groups, giving 80% power to detect a treatment effect size of 20% compared to a baseline response of 5% at a significance level of 0.05 (Clayton, J. A. & Collins, F. S. Nature 509, 282-283 (2014); Andrews, N. A. et al. Pain 157, 901-909, (2016)). Numbers required to achieve statistical power for the ADME studies were determined by G.Power3.1.

As discussed in EXAMPLES 1-4, the peptides of the present disclosure prevents fat cell accumulation through its reductive effect on fructose levels in the body, which in turn impedes fatty acid synthesis. This is particularly significant during the ongoing obesity epidemic and in a modern world of high-sugar diets and increased consumption of fructose. Maintaining low fructose levels has a dual beneficial effect and not only reduces fat cell accumulation but also helps prevent collagen degradation. Collagen plays an important role in retaining healthy and smooth skin after long-time, inevitable exposure to damaging factors, such as sunlight and pollution. Thus, sustaining high levels of collagen and low levels of fructose contribute to the elasticity and youthfulness of the skin and ultimately decelerates the aging process.

In addition, it is predicted that the method of application to the skin will provide an effect similar to the intraperitoneal and subcutaneous injection methods we have used in our studies. In the end, the treatment essentially plays the same role in reducing cell accumulation regardless of the injection or application route. After application to the skin, the treatment would enter the skin layer and would be expected to show the effect of reducing fat cell accumulation at the fat cell level.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. A cosmetic composition comprising one or more peptides having an amino acid sequence selected from the group consisting of KGGRAKD (SEQ ID NO:1), KGG, GGR, GRA, RAK, AKD, DKA, KAR, ARG, RGG, GGK, DKARGGK (SEQ ID NO:2), ACTGSTQHQGGGSEHGAMEI (SEQ ID NO: 3), ACSSSPSKHCGGGSESPLKRQG (SEQ ID NO: 4), and ACTGSTQHQGGGSESPLKRQG (SEQ ID NO: 5) or a variant or mimetic thereof.

2. The cosmetic composition of claim 1, wherein the peptide is cyclized.

3. The cosmetic composition of claim 2, wherein the peptide is cyclized via addition of a cysteine to each end of the peptide.

4. The cosmetic composition of claim 1, wherein the peptide is modified.

5. The cosmetic composition of claim 1, further comprising a cosmetically acceptable topical carrier.

6. The cosmetic composition of claim 5, further comprising one or more additional cosmetic agents.

7. The cosmetic composition of claim 1, wherein the cosmetic composition comprises two or more of the peptides.

8. The cosmetic composition of claim 1, wherein the peptide is RAK or GGR.

9. The cosmetic composition of claim 1, wherein the cosmetic composition is for use in reducing a level of glucose, fructose, sucrose or polysaccharide in adipocytes or skin cells.

10. The cosmetic composition of claim 1, wherein the cosmetic composition is for use in preventing collagen degradation.

11. The cosmetic composition of claim 1, wherein the cosmetic composition is for use in rejuvenating skin.

12. The cosmetic composition of claim 1, wherein the cosmetic composition is formulated in the form of a cream, a lotion, a sunscreen product, an ointment, a spray, a powder, a tanning product, or a colored cosmetic product.

13. A patch comprising the cosmetic composition of claim 1.

14. The patch of claim 13, wherein the patch is in the form of a microneedle patch, or a hyaluronic acid patch.

15. A method of improving skin rejuvenation, comprising:
applying the cosmetic composition of claim 1 to a skin of a subject in need thereof.

16. The method of claim 15, wherein the applying the cosmetic composition reduces a level of glucose, fructose, sucrose or polysaccharide in adipocytes or skin cells of the subject.

17. The method of claim 15, wherein the applying the cosmetic composition prevents collagen degradation in the subject.

18. A method of improving skin rejuvenation, comprising:
applying the patch of claim 13 to a skin of a subject in need thereof.

19. The method of claim 18, wherein the applying the patch reduces a level of glucose, fructose, sucrose or polysaccharide in adipocytes or skin cells of the subject.

20. The method of claim 18, wherein the applying the patch prevents collagen degradation in the subject.
